(19) United States
(12) Patent Application Publication
(10) Pub. No.: US 2019/0254550 A1
(43) Pub. Date: Aug. 22, 2019

(54) WET ELECTRODE FOR ABDOMINAL FETAL ELECTROCARDIOGRAM DETECTION

(71) Applicant: Nuvo Group Ltd., Tel Aviv (IL)

(72) Inventors: Ilya Divinsky, Ramat Gan (IL); Andrew Moonin, Ariel (IL); Oren Oz, Modiin (IL)

(21) Appl. No.: 16/279,768

(22) Filed: Feb. 19, 2019

Related U.S. Application Data
(60) Provisional application No. 62/632,136, filed on Feb. 19, 2018.

(51) Int. Cl. A61B 5/0448; A61B 5/00; A61B 5/0408; A61B 5/0416

(52) U.S. Cl. CPC A61B 5/0448; A61B 5/0006; A61B 5/04087; A61B 5/0416; A61B 5/04085; A61B 5/6823; A61B 2562/0217; A61B 2562/0215; A61B 5/6831; A61B 2562/0204; A61B 5/6804

(57) ABSTRACT

An electrode includes a cutaneous contact for sensing electrocardiogram signals from a pregnant human subject, the electrocardiogram signals containing fetal electrocardiogram signals; a connector in contact with the cutaneous contact for transmission of the electrocardiogram signals from the cutaneous contact to a destination; and a cushion including a cavity, the cushion configured such that the cavity faces the pregnant human subject when in use, the cutaneous contact being coupled to the cushion such that the cutaneous contact is positioned within the cavity, the cushion and the cutaneous contact configured to allow the cutaneous contact to be an electrical interface with skin of the subject when the electrode is in use, the cavity being configured to receive and retain therein an amount of a conductive wetting substance sufficient to provide a skin-electrode impedance of less than 150 kΩ when the electrocardiogram signals are at frequencies of 10 Hz or less.

### CROSS-REFERENCE TO RELATED APPLICATION

This is a U.S. utility patent application relating to and claiming the benefit of commonly-owned, co-pending U.S. Provisional Patent Application No. 62/632,136, filed Feb. 19, 2018, entitled “WET ELECTRODE FOR ABDOMINAL FETAL ELECTROCARDIOGRAM DETECTION,” the contents of which are incorporated herein by reference in their entirety.

### FIELD OF THE INVENTION

The invention relates generally to electrodes suitable for use in detecting bio-potential. More particularly, the invention relates to electrodes suitable for EMG detection, EHG detection, EEG detection, and the like. More particularly, the invention relates to electrodes suitable for use in fetal heart rate monitoring systems, maternal uterine activity monitoring systems, and other types of pregnancy monitoring systems.

### BACKGROUND

Monitoring fetal cardiac electrical activity can be useful to determine of the health of a fetus during pregnancy.

### SUMMARY

In an embodiment, an electrode includes (a) a cutaneous contact for sensing electrocardiogram signals from a pregnant human subject, the electrocardiogram signals containing fetal electrocardiogram signals; (b) a connector in electrical contact with the cutaneous contact for transmission of the electrocardiogram signals from the cutaneous contact to a destination; and (c) a cushion including a cavity, the cushion being configured such that the cavity faces the pregnant human subject when the electrode is in use, the cutaneous contact is coupled to the cushion such that the cutaneous contact is positioned within the cavity, the cushion and the cutaneous contact configured to allow a surface of the cutaneous contact to be an electrical interface with a skin of the pregnant human subject when the electrode is in use, and the cavity is configured so as to receive and retain therein an amount of a conductive wetting substance that is sufficient to provide a skin-electrode impedance of less than 150 kΩ when the electrocardiogram signals are at frequencies of 10 Hz or less.

In an embodiment, the cutaneous contact includes silver. In an embodiment, the skin-electrode impedance is between 5 kΩ and 150 kΩ at frequencies of 10 Hz and below. In an embodiment, the electrode provides a signal-to-noise ratio of fetal electrocardiogram signals that is less than 50 dB.

In an embodiment, the electrode also includes a button snap stud having a flange portion; and a button snap post having a flange portion, the button snap post being configured to snap into the button snap stud, wherein the cushion includes a lip surrounding a central bore, the central bore being sized such that the flange portion of the button snap stud and the flange portion of the button snap post cannot pass through the central bore, wherein the button snap post is snapped into the button snap stud in respective positions such that flange portion of the button snap post and the flange portion of the button snap stud are positioned on opposite sides of the lip of the cushion so as to fix the button snap post and the button snap stud to the cushion, and wherein the cutaneous contact is retained by the button snap post. In an embodiment, the electrode also includes a frame comprising a plastic material, wherein the cushion encompasses the frame.

In an embodiment, the cavity is circular and has an opening diameter at an opening where the cushion contacts the skin of the pregnant human subject, and the opening diameter is between 12 mm and 17 mm. In an embodiment, the cavity has a base diameter at a base opposite the opening, and wherein the base diameter is between 9 mm and 11 mm. In an embodiment, the cavity has a depth of between 0.2 mm and 4.5 mm. In an embodiment, the electrode meets at least one criterion of an ANSI/AAMI EC12:2000/®2015 standard for Disposable ECG Electrodes.

In an embodiment, a kit includes a conductive wetting substance and at least one electrode configured to detect fetal electrocardiogram signals through the use of the wetting substance, the electrode including (a) a cutaneous contact for sensing fetal electrocardiogram signals from a pregnant human subject; (b) a connector in electrical contact with the cutaneous contact for transmission of a signal from the cutaneous contact to a destination; and (c) a cushion including a cavity, the cushion being configured such that the cavity faces the pregnant human subject when the electrode is in use, wherein the cutaneous contact is coupled to the cushion such that the cutaneous contact is positioned within the cavity, the cushion and the cutaneous contact configured to allow a surface of the cutaneous contact to be in electrical communication with the skin of the pregnant human subject when the electrode is in use, and wherein the cavity is configured so as to receive and retain therein an amount of the conductive wetting substance sufficient so as to provide a skin-electrode impedance of less than 150 kΩ at frequencies of 10 Hz and below.

In an embodiment, the wetting substance is a conductive gel. In an embodiment, the conductive gel has an electrical length admittance of between 3.6 and 313 μS/cm at a frequency of 1 Hz

In an embodiment, the wetting substance is a conductive paste. In an embodiment, the conductive paste has an electrical length admittance of between 250 and 300 μS/cm at a frequency of 1 Hz

In an embodiment, a garment includes at least one pair of electrodes, each of the electrodes being configured to detect fetal electrocardiogram signals through the use of a wetting substance, each of the electrodes including (a) a cutaneous contact for sensing fetal electrocardiogram signals from a pregnant human subject; (b) a connector in electrical contact with the cutaneous contact for transmission of a signal from the cutaneous contact to a destination; and (c) a cushion including a cavity, the cushion being configured such that the cavity faces the pregnant human subject when the garment is worn by the pregnant human subject, wherein the cutaneous contact is coupled to the cushion such that the cutaneous contact is positioned within the cavity, the cushion and the cutaneous contact configured to allow a surface of the cutaneous contact to be in electrical communication with the skin of the pregnant human subject when the electrode is in use, and wherein the cavity is configured so as to receive and retain therein an amount of a conductive wetting substance sufficient so as to provide a skin-electrode impedance of less than 150 kΩ at frequencies of 10 Hz and below.

In an embodiment, the garment includes a belt. In an embodiment, the garment also includes at least one acoustic sensor. In an embodiment, the at least one pair of includes comprises eight electrodes. In an embodiment, the electrodes are positioned within the garment such that, when the garment is worn by the pregnant human subject, the electrodes are positioned in a circumferential arrangement around a uterus of the pregnant human subject.

In one embodiment, the present invention provides an electrode configured to detect fetal electrocardiogram signals, comprising:
a) a cutaneous contact for sensing fetal electrocardiogram signals from a pregnant human subject;
b) a connector in electrical contact with the cutaneous contact for connection to a lead wire; and
c) a cushion coupled to the cutaneous contact, the cushion including a recess and configured such that the cutaneous contact is positioned within the recess,
   wherein, the cushion and the cutaneous contact are configured to allow a surface of the cutaneous contact to be in electrical interface (e.g., communication) with the skin of the pregnant human subject.

In one embodiment, the cutaneous contact is configured to have skin-electrode impedance of greater than 150 kΩ.

In one embodiment, the cutaneous contact is configured to have skin-electrode impedance of less than 150 kΩ.

In one embodiment, the cutaneous contact is configured to have skin-electrode impedance of between 5 to 150 kΩ.

In one embodiment, the signal to noise ratio of the fetal electrocardiogram signals is between −20 dB and 50 dB.

In one embodiment, the signal to noise ratio of the fetal electrocardiogram signals is between 0 dB and 50 dB.

In one embodiment, the signal to noise ratio of the fetal electrocardiogram signals is less than 50 dB.

In one embodiment, the present invention provides a garment, comprising:

at least one pair of electrodes,
wherein the at least one pair of electrodes are configured, when the garment is worn by the pregnant human subject, such that the individual electrodes of the at least one electrode pair encircle the uterus of the pregnant human subject, and
wherein the individual electrodes of the at least one electrode pair comprise:
   a) a cutaneous contact for sensing fetal electrocardiogram signals from a pregnant human subject;
   b) a connector in electrical contact with the cutaneous contact for connection to a lead wire; and
   c) a cushion coupled to the cutaneous contact, the cushion including a recess and configured such that the cutaneous contact is positioned within the recess,
   wherein, the cushion and the cutaneous contact are configured to allow a surface of the cutaneous contact to be in electrical interface (e.g., communication) with the skin of the pregnant human subject.
wherein cardiac electrical activity data is recorded from the at least one sensor pair.

### BRIEF DESCRIPTION OF THE DRAWINGS

FIG. 1 shows an exploded view of elements of an electrode according to an embodiment of the present invention.

FIG. 2 shows a cross-sectional view of an electrode according to a first embodiment of the present invention.

FIG. 3 shows a flowchart of a method for making the electrode of FIG. 3.

FIG. 4 shows a cross-sectional view of an electrode according to a second embodiment of the present invention.

FIG. 5 shows a flowchart of a method for making the electrode of FIG. 4.

FIG. 6 shows a front view of the positions of the ECG sensor pairs on the abdomen of a pregnant woman according to some embodiments of the present invention.

FIG. 7 shows a side view of the positions of the ECG sensor pairs on the abdomen of a pregnant woman according to some embodiments of the present invention.

FIG. 8 shows a representation of a system suitable for use in fetal heart rate monitoring systems according to some embodiments of the present invention.

FIG. 9 shows a garment according to some embodiments of the present invention.

FIG. 10 shows the garment of FIG. 9 as worn by a pregnant human subject.

FIG. 11 shows a diagram of an equivalent circuit for an electrode.

FIG. 12 shows a first set of recorded test data comparing an exemplary experimental electrode to a reference electrode.

FIG. 13 shows a second set of recorded test data comparing an exemplary experimental electrode to a reference electrode.

FIG. 14 shows a third set of recorded test data comparing an exemplary experimental electrode to a reference electrode.

FIG. 15 shows a cross-sectional view of an electrode according to a third embodiment of the present invention.

FIG. 16 shows a flowchart of a method for making the electrode of FIG. 15.

### DETAILED DESCRIPTION

For clarity of disclosure, and not by way of limitation, the detailed description of the invention is divided into the following subsections that describe or illustrate certain features, embodiments or applications of the present invention.

As used herein the term “contact region” encompasses the contact area between the skin of a pregnant human subject and cutaneous contact i.e. the surface area through which current flow can pass between the skin of the pregnant human subject and the cutaneous contact.

In some embodiments, the present invention provides an electrode for recording electrocardiogram data. In some embodiments, the electrode includes a contact mounted to a frame and an elastomeric cushion formed around the frame and shaped such that the contact is positioned within a recess of the cushion. In some embodiments, the contact includes silver and silver chloride.

In some embodiments, the present invention provides a system for detecting, recording and analyzing cardiac electrical activity data from a pregnant human subject. In some embodiments, a plurality of electrodes configured to detect fetal electrocardiogram signals is used to record the cardiac activity data. In some embodiments, a plurality of electrodes configured to detect fetal electrocardiogram signals and a plurality of acoustic sensors are used to record the cardiac activity data. In some embodiments, a plurality of electrodes is configured to detect maternal uterine activity. In some embodiments, a plurality of electrodes is configured to detect fetal activity. In some embodiments, a plurality of electrodes is configured to detect maternal respiration rate.

In some embodiments, a plurality of electrodes configured to detect fetal electrocardiogram signals are attached to the abdomen of the pregnant human subject. In some embodiments, the plurality of electrodes configured to detect fetal electrocardiogram signals are directly attached to the abdomen. In some embodiments, the plurality of electrodes configured to detect fetal electrocardiogram signals are incorporated into an article, such as, for example, a belt, a patch, and the like, and the article is worn by, or placed on, the pregnant human subject.

Without intending to be limited to any particular theory, in some embodiments, the three-dimensional shape of the electrode affects the performance. For example, a curved profile without sharp angles is likely to prevent abrupt changes in the electrical field generated by the cutaneous contact, or flow of current from the cutaneous contact to the lead wire.

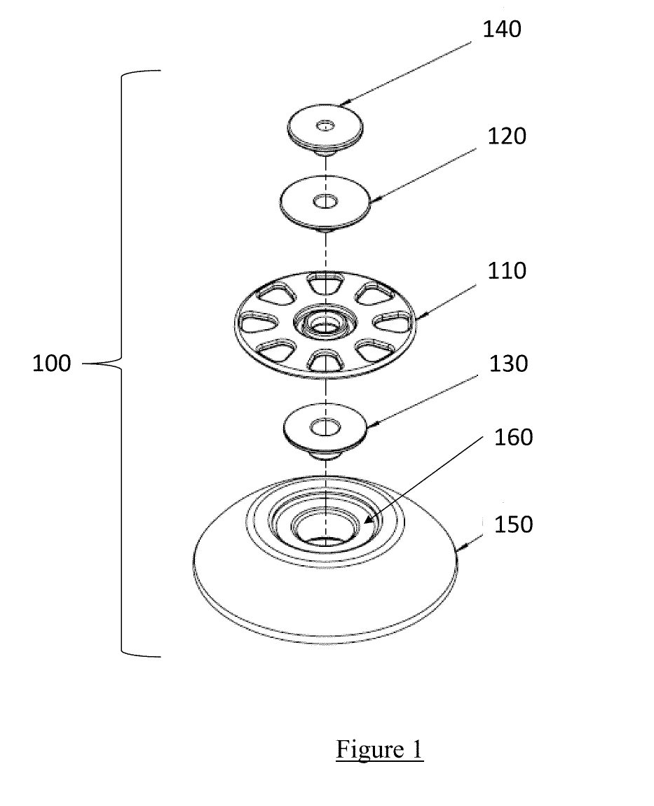

FIG. 1 shows an exploded view of elements of an electrode 100 according to some embodiments of the present invention. In the embodiment shown in FIG. 1, the electrode 100 includes a frame 110. In some embodiments, the frame 110 is made from a plastic material. In some embodiments, the frame 110 is made from a polycarbonate plastic. In some embodiments, the frame 110 is made from a nylon plastic. In some embodiments, the frame 110 is made from acrylonitrile butadiene styrene (“ABS”). In some embodiments, the frame 110 is made from a metal. In some embodiments, the frame 110 is made from a metal including aluminum. In some embodiments, the frame 110 is made from a sheet metal. In some embodiments, the frame 110 is made from a die-cast metal. In some embodiments, the frame 110 is made from an extruded metal. In some embodiments, the frame 110 has a circular profile, a central bore, and a plurality of perforations circumferentially arrayed near a perimeter thereof.

In some embodiments, the electrode 100 includes a button snap post 120 and a button snap stud 130. In some embodiments, the button snap post 120 is configured to snap into and engage the button snap stud 130. In some embodiments, the button snap post 120 has a post portion that is sized so as to pass through the central bore of the frame 110 and a flange portion that is sized so as to be unable to pass through the central bore of the frame 110. In some embodiments, the button snap stud 130 includes a post portion including a central bore that is configured to receive the post portion of the button snap post 120 and a flange portion that is sized so as to be unable to pass through the central bore of the frame 110. In some embodiments, the button snap post 120 and the button snap stud 130 are configured so as to be secured to opposite sides of the frame 110 when the post portion of the button snap post 120 is inserted through the central bore of the frame 110 and engaged with the central bore of the button snap stud 130. In some embodiments, the button snap stud 130 is configured to be attached to an underlying structure (e.g., a garment, such as a belt). In some embodiments, the button snap stud 130 is configured to attach to a lead wire or other signal conducting element. In some embodiments, the button snap post 120 and the button snap stud 130 are made from conductive and non-corrosive materials. In some embodiments, the button snap post 120 and the button snap stud 130 are made the same conductive and non-corrosive material. In some embodiments, the button snap post 120 and the button snap stud 130 are made from different conductive and non-corrosive materials. In some embodiments, the button snap post 120 and the button snap stud 130 are made from stainless steel. In some embodiments, the button snap post 120 and the button snap stud 130 are made from AISI type 304 stainless steel.

In some embodiments, the electrode 100 includes a contact 140 that is configured to have a wetting substance (e.g., a gel or a paste) applied thereto, and to be placed in contact with a patient's skin at an appropriate location in order to sense an ECG signal. In some embodiments, the electrode 100 includes a contact material. In some embodiments, the contact material includes silver (Ag). In some embodiments, the contact material includes silver chloride (AgCl). In some embodiments, the contact material includes both Ag and AgCl (Ag/AgCl). In some embodiments, the contact material includes another material having suitable properties (e.g., electrical conductivity, half-cell potential, etc.) to operate as described herein. In some embodiments, the electrode 100 includes a base material coated by the contact material. In some embodiments, the base material is a polymer material. In some embodiments, the base material is a plastic material. In some embodiments, the base material is a polycarbonate plastic. In some embodiments, the base material is a nylon plastic. In some embodiments, the base material is Acrylonitrile Butadiene Styrene (ABS). In some embodiments, the base material is the polycarbonate plastic commercialized under the trademark MAKROLON® 2458 by Bayer MaterialScience AG of Leverkusen, Germany. In some embodiments, the contact 140 includes an attachment portion and a contact portion. In some embodiments, the attachment portion is configured to snap into the button snap post 120. In some embodiments, the contact portion is configured to contact a person's skin as described above.

In some embodiments, the wetting substance is a gel. In some embodiments, the gel is a hydrogel. In some embodiments, the gel is water-soluble. In some embodiments, the gel is conductive. In some embodiments, the gel is configured to wet the skin and thereby reduce skin resistance. In some embodiments, the gel is non-conductive. In some embodiments, the gel is salt-free. In some embodiments, the gel is chloride-free. In some embodiments, the gel is high-viscosity and adhesive. In some embodiments, the gel is sufficiently high-viscosity and adhesive so as to reduce motion artifacts. In some embodiments, peel-off resistance of the gel is between 0.5 N and 5 N. In some embodiments, the gel is that commercialized under the trade name SPECTRA 360 by Parker Laboratories of Fairfield, N.J. In some embodiments, the gel is that commercialized under the trade name COMFORT GEL M807 by R&D Medical Products of Lake Forest, Calif. In some embodiments, the gel sets an impedance of less than 150 kΩ at frequencies of 10 Hz and below between skin and electrode due to the conductive effect on the epidermal stratum corneum. In some embodiments, the gel sets an impedance of less than 150 kΩ at frequencies of between 0.05 Hz and 150 Hz between skin and electrode due to the conductive effect on the epidermal stratum corneum. In some embodiments, the gel has an electrical length admittance of between 3.6 and 313 μS/cm at a frequency of 1 Hz. In some embodiments, the gel provides an effective electrode area (“EEA”) of between 1.75 and 2.75 cm². In some embodiments, the gel provides an EEA of between 0.75 and 2.75 cm². In some embodiments, the impedance measured between a pair of electrodes connected gel to gel is less than or equal to 3 kΩ, which is a pass criterion of the ANSI/AAMI EC12:2000/®2015 standard for Disposable ECG Electrodes. In some embodiments, the DC Offset Voltage of a pair of electrodes connected gel to gel after one minute of stabilization time is less than or equal to 100 mV, which is a pass criterion of the ANSI/AAMI EC12:2000/®2015 standard for Disposable ECG Electrodes. In some embodiments, the Combined Offset Instability and Internal Noise of a pair of electrodes connected gel to gel, after a one-minute stabilization period, measured in the passband of 0.15 to 100 Hz for five minutes is less than or equal to 150 μV, which is a pass criterion of the ANSI/AAMI EC12:2000/®2015 standard for Disposable ECG Electrodes. In some embodiments, the DC Voltage Offset of a pair of electrodes connected gel to gel, after the end of a monitoring session, is less than or equal to 150 mV, which is a pass criterion of the ANSI/AAMI EC12:2000/®2015 standard for Disposable ECG Electrodes.

In some embodiments, the wetting substance is a paste. In some embodiments, a paste provides better skin contact during long-term monitoring as compared to a wet gel that may dry out during use. In some embodiments, the paste is water-soluble. In some embodiments, the paste is conductive. In some embodiments, the paste has similar or better conductivity properties as compared to the conductive gels discussed above. In some embodiments, the paste maintains its performance over a period of time better than gel as it is more viscous and loses water more slowly. In some embodiments, the paste is non-conductive. In some embodiments, the paste contains conductive carbon. In some embodiments, the paste is adhesive. In some embodiments, the paste is sufficiently adhesive so as to reduce motion artifacts. In some embodiments, the paste is salt-free. In some embodiments, the paste is chloride-free. In some embodiments, the paste includes one or more of the following ingredients: polyoxyethylene (20) cetyl ether, water, glycerin, calcium carbonate, 1,2-propanediol, potassium chloride, gelwhite, sodium chloride, polyoxyethylene (20) sorbitol, methylparaben, and propylparaben. In some embodiments, the paste is that commercialized under the trade name TEN20 by Weaver and Company of Aurora, Colo. In some embodiments, the paste has an impedance of less than 150 kΩ at frequencies of 10 Hz and below due to the conductive effect on the epidermal stratum corneum. In some embodiments, the paste has an impedance of less than 150 kΩ at frequencies of between 0.05 Hz and 150 Hz due to the conductive effect on the epidermal stratum corneum. In some embodiments, the paste has an electrical conductivity of between 250 and 300 μS/cm at a frequency of 1 Hz. In some embodiments, the paste has an electrical conductivity of 282 μS/cm at a frequency of 1 Hz. In some embodiments, the paste has an EEA of between 4.5 and 6.5 cm².

In some embodiments, the impedance measured between a pair of electrodes connected paste to paste is less than or equal to 3 kΩ, which is a pass criterion of the ANSI/AAMI EC12:2000/®2015 standard for Disposable ECG Electrodes. In some embodiments the DC Offset Voltage of a pair of electrodes connected paste to paste after one minute of stabilization time is less than or equal to 100 mV, which is a pass criterion of the ANSI/AAMI EC12:2000/®2015 standard for Disposable ECG Electrodes. In some embodiments the Combined Offset Instability and Internal Noise of a pair of electrodes connected paste to paste, after one minute of stabilization time, measured in the passband of 0.15 to 100 Hz for five minutes is less than or equal to 150 μV, which is a pass criterion of the ANSI/AAMI EC12:2000/®2015 standard for Disposable ECG Electrodes. In some embodiments, the DC Voltage Offset of a pair of electrodes connected paste to paste, after a monitoring session ends, is less than or equal to 150 mV, which is a pass criterion of the ANSI/AAMI EC12:2000/®2015 standard for Disposable ECG Electrodes.

In some embodiments, the electrode 100 includes a cushion 150. In some embodiments, the cushion 150 is made from an elastomeric material. In some embodiments, the cushion 150 is made from silicone. In some embodiments, the cushion 150 is configured to engage the frame 110 and be fixed thereto. In some embodiments, the cushion 150 is overmolded to the frame 110. In some embodiments, the cushion 150 is configured (e.g., sized, shaped, and made from an appropriate material) so as to enable the electrode 100 to be comfortably positioned adjacent the wearer's skin. In some embodiments, the cushion 150 includes a first side that is generally convex and a second side that is generally concave. In some embodiments, the second side includes a cavity 160 that is configured to receive and retain the gel therein. In some embodiments, the cavity 160 is sized and shaped to receive and retain the gel therein. In some embodiments, the size of the cavity 160 includes an opening diameter at the opening where the cavity 160 meets the skin and an inner diameter at the opposite end of the cavity 160 near the contact 140. In some embodiments, the opening diameter and the inner diameter are sufficiently large so as to hold an effective amount of the gel, but are sufficiently small so as to prevent the gel from running out of the cavity 160 or quickly drying.

In some embodiments, the opening diameter is between 12 and 17 mm. In some embodiments, the opening diameter is between 12 and 16 mm. In some embodiments, the opening diameter is between 12 and 15 mm. In some embodiments, the opening diameter is between 12 and 14 mm. In some embodiments, the opening diameter is between 12 and 13 mm. In some embodiments, the opening diameter is between 13 and 17 mm. In some embodiments, the opening diameter is between 13 and 16 mm. In some embodiments, the opening diameter is between 13 and 15 mm. In some embodiments, the opening diameter is between 13 and 14 mm. In some embodiments, the opening diameter is between 14 and 17 mm. In some embodiments, the opening diameter is between 14 and 16 mm. In some embodiments, the opening diameter is between 14 and 15 mm. In some embodiments, the opening diameter is between 15 and 17 mm. In some embodiments, the opening diameter is between 15 and 16 mm. In some embodiments, the opening diameter is between 16 and 17 mm. In some embodiments, the opening diameter is about 12 mm. In some embodiments, the opening diameter is about 13 mm. In some embodiments, the opening diameter is about 14 mm. In some embodiments, the opening diameter is about 15 mm. In some embodiments, the opening diameter is about 16 mm. In some embodiments, the opening diameter is about 17 mm.

In some embodiments, the inner diameter is between 9.0 mm and 11.0 mm. In some embodiments, the inner diameter is between 9.0 mm and 10.5 mm. In some embodiments, the inner diameter is between 9.0 mm and 10.0 mm. In some embodiments, the inner diameter is between 9.0 mm and 9.5 mm. In some embodiments, the inner diameter is between 9.5 mm and 11.0 mm. In some embodiments, the inner diameter is between 9.5 mm and 10.5 mm. In some embodiments, the inner diameter is between 9.5 mm and 10.0 mm. In some embodiments, the inner diameter is between 10.0 mm and 11.0 mm. In some embodiments, the inner diameter is between 10.0 mm and 10.5 mm. In some embodiments, the inner diameter is between 10.5 mm and 11.0 mm. In some embodiments, the inner diameter is about 9.0 mm. In some embodiments, the inner diameter is about 9.5 mm. In some embodiments, the inner diameter is about 10.0 mm. In some embodiments, the inner diameter is about 10.5 mm. In some embodiments, the inner diameter is about 11.0 mm.

In some embodiments, the size of the cavity 160 includes a depth. In some embodiments, the depth is sufficiently large so as to hold an effective amount of the gel, but is sufficiently small so as to prevent the gel from running out of the cavity or quickly drying. In some embodiments, the depth is between 0.2 mm and 4.5 mm. In some embodiments, the depth is between 0.2 mm and 4.0 mm. In some embodiments, the depth is between 0.2 mm and 3.5 mm. In some embodiments, the depth is between 0.2 mm and 3.0 mm. In some embodiments, the depth is between 0.2 mm and 2.5 mm. In some embodiments, the depth is between 0.2 mm and 2.0 mm. In some embodiments, the depth is between 0.2 mm and 1.5 mm. In some embodiments, the depth is between 0.2 mm and 0.5 mm. In some embodiments, the depth is between 0.5 mm and 4.5 mm. In some embodiments, the depth is between 0.5 mm and 4.0 mm. In some embodiments, the depth is between 0.5 mm and 3.5 mm. In some embodiments, the depth is between 0.5 mm and 3.0 mm. In some embodiments, the depth is between 0.5 mm and 2.5 mm. In some embodiments, the depth is between 0.5 mm and 2.0 mm. In some embodiments, the depth is between 0.5 mm and 1.5 mm. In some embodiments, the depth is between 0.5 mm and 1.0 mm. In some embodiments, the depth is between 1.0 mm and 4.5 mm. In some embodiments, the depth is between 1.0 mm and 4.0 mm. In some embodiments, the depth is between 1.0 mm and 3.5 mm. In some embodiments, the depth is between 1.0 mm and 3.0 mm. In some embodiments, the depth is between 1.0 mm and 2.5 mm. In some embodiments, the depth is between 1.0 mm and 2.0 mm. In some embodiments, the depth is between 1.0 mm and 1.5 mm. In some embodiments, the depth is between 1.5 mm and 4.5 mm. In some embodiments, the depth is between 1.5 mm and 4.0 mm. In some embodiments, the depth is between 1.5 mm and 3.5 mm. In some embodiments, the depth is between 1.5 mm and 3.0 mm. In some embodiments, the depth is between 1.5 mm and 2.5 mm. In some embodiments, the depth is between 1.5 mm and 2.0 mm. In some embodiments, the depth is between 2.0 mm and 4.5 mm. In some embodiments, the depth is between 2.0 mm and 4.0 mm. In some embodiments, the depth is between 2.0 mm and 3.5 mm. In some embodiments, the depth is between 2.0 mm and 3.0 mm. In some embodiments, the depth is between 2.0 mm and 2.5 mm. In some embodiments, the depth is between 2.5 mm and 4.5 mm. In some embodiments, the depth is between 2.5 mm and 4.0 mm. In some embodiments, the depth is between 2.5 mm and 3.5 mm. In some embodiments, the depth is between 2.5 mm and 3.0 mm. In some embodiments, the depth is between 3.0 mm and 4.5 mm. In some embodiments, the depth is between 3.0 mm and 4.0 mm. In some embodiments, the depth is between 3.0 mm and 3.5 mm. In some embodiments, the depth is between 3.5 mm and 4.5 mm. In some embodiments, the depth is between 3.5 mm and 4.0 mm. In some embodiments, the depth is between 0.0 mm and 4.5 mm. In some embodiments, the depth is about 0.2 mm. In some embodiments, the depth is about 0.5 mm. In some embodiments, the depth is about 1.0 mm. In some embodiments, the depth is about 1.5 mm. In some embodiments, the depth is about 2.0 mm. In some embodiments, the depth is about 2.5 mm. In some embodiments, the depth is about 3.0 mm. In some embodiments, the depth is about 3.5 mm.

In some embodiments, the electrode 100 includes a printed circuit board. In some embodiments, the printed circuit board is configured to interface the electrode 100 with a lead wire. In some embodiments, the printed circuit board is further configured to perform additional functions, such as, for example, signal filtering, or pre-amplification.

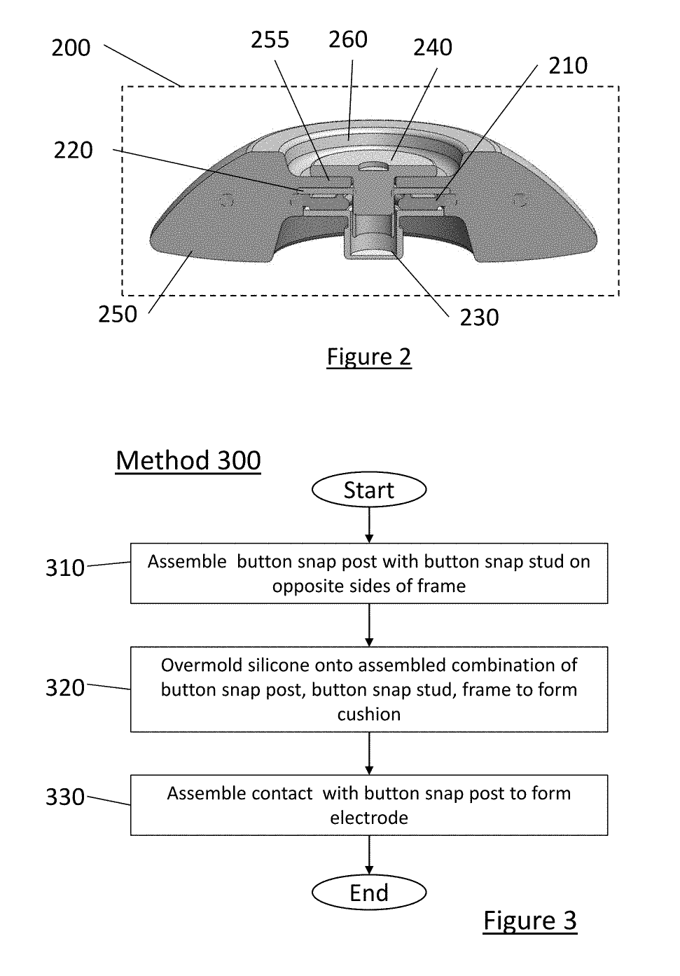

FIG. 2 shows a cross-sectional view of an electrode 200 according to an embodiment of the present invention. In some embodiments, the electrode 200 includes a frame 210, a button snap post 220, a button snap stud 230, a contact 240, and a cushion 250, which are substantially similar to the frame 110, the button snap post 120, the button snap stud 130, the contact 140, and the cushion 150 of the electrode 100 other than as described hereinafter. In some embodiments, the cushion 250 includes a lip 255 and a cavity 260. In some embodiments, the elements of the electrode 200 are assembled such that the lip 255 of the cushion 250 is positioned between the contact portion of the contact 240 and the flange portion of the button snap post 220.

FIG. 3 shows an exemplary embodiment of a method 300 for making an electrode such as the electrode 200 of FIG. 2. In step 310, the button snap post 220 and the button snap stud 230 are assembled together to either side of the frame 210, as discussed above with reference to FIG. 1. In step 320, an elastomeric material (e.g., silicone) is overmolded to the assembled combination of the button snap post 220, the frame 210, and the button snap stud 230 to form the cushion 250, which includes the lip 255. In step 330, the contact 240 is assembled to the button snap post 220, thereby forming the assembled electrode 200.

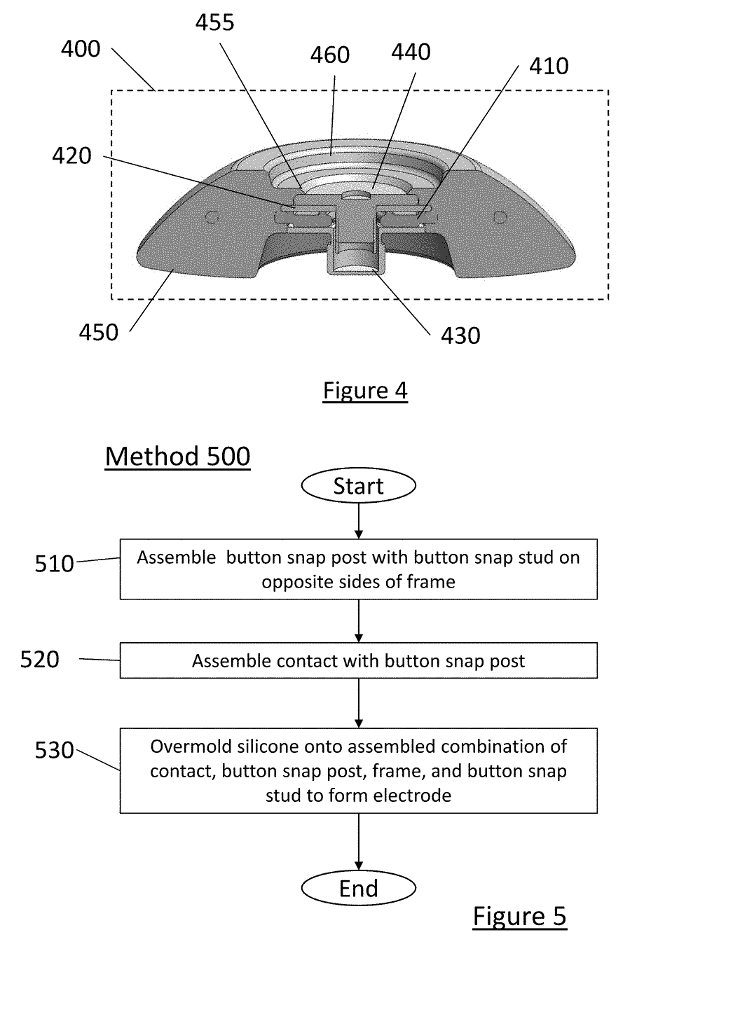

FIG. 4 shows a cross-sectional view of an electrode 400 according to an embodiment of the present invention. In some embodiments, the electrode 400 includes a frame 410, a button snap post 420, a button snap stud 430, a contact 440, and a cushion 450, which are substantially similar to the frame 110, the button snap post 120, the button snap stud 130, the contact 140, and the cushion 150 of the electrode 100 other than as described hereinafter. In some embodiments, the cushion 450 includes a lip 455 and a cavity 460. In some embodiments, the elements of the electrode 400 are assembled such that the contact portion of the contact 440 is positioned between the lip 455 of the cushion 450 and the flange portion of the button snap post 420.

FIG. 5 shows an exemplary embodiment of a method 500 for making an electrode such as the electrode 400 of FIG. 4. In step 510, the button snap post 420 and the button snap stud 430 are assembled together to either side of the frame 410, as discussed above with reference to FIG. 1. In step 520, the contact 440 is assembled to the button snap post 420. In step 530, an elastomeric material (e.g., silicone) is overmolded to the assembled combination of the contact 440, the button snap post 420, the frame 410, and the button snap stud 430 to form the cushion 450, which includes the lip 455. The formation of the cushion 450 results in the finished electrode 400.

FIG. 15 shows a cross-sectional view of an electrode 1500 according to an embodiment of the present invention. In some embodiments, the electrode 1500 includes a button snap post 1520, a button snap stud 1530, a contact 1540, and a cushion 1550, which are substantially similar to the button snap post 120, the button snap stud 130, the contact 140, and the cushion 150 of the electrode 100 other than as described hereinafter. In some embodiments, the cushion 1550 includes a lip 1555 and a cavity 1560. In some embodiments, the elements of the electrode 1500 are assembled such that the lip 1555 of the cushion 1550 is positioned between the flange portion of the button snap stud 1530 and the flange portion of the button snap post 1520.

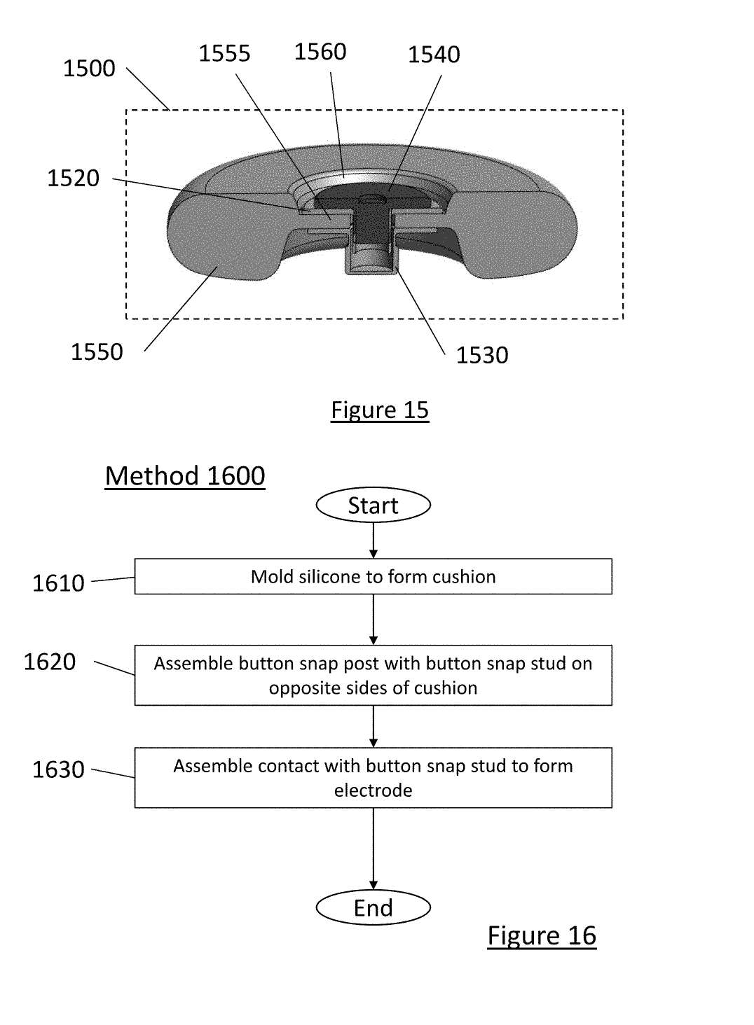

FIG. 16 shows an exemplary embodiment of a method 1600 for making an electrode such as the electrode 1500 of FIG. 15. In step 1510, an elastomeric material (e.g., silicone) is molded to form the cushion 1550, which includes the lip 1555. In step 1520, the button snap post 1520 and the button snap stud 1530 are assembled together to either side of the cushion 1550 so as to enclose the lip 1555 of the cushion 1550. In step 1530, the contact 1540 is assembled to the button snap post 1520, thereby forming the assembled electrode 1500.

In some embodiments, the cushion (e.g., the cushion 150, the cushion 250, the cushion 450, or the cushion 1550) has a diameter ranging from 20 to 50 mm. In some embodiments, the cushion has a diameter of 20 mm. In some embodiments, the cushion has a diameter of 25 mm. In some embodiments, the cushion has a diameter of 30 mm. In some embodiments, the cushion has a diameter of 35 mm. In some embodiments, the cushion has a diameter of 40 mm. In some embodiments, the cushion has a diameter of 45 mm. In some embodiments, the cushion has a diameter of 50 mm.

In some embodiments, the cushion is configured to generate a profile without sharp angles which are likely affect performance of the electrode. In some embodiments, the cushion has a diameter ranging from 15 to 38 mm. In some embodiments, the cushion has a diameter of 15 mm. In some embodiments, the cushion has a diameter of 20 mm. In some embodiments, the cushion has a diameter of 25 mm. In some embodiments, the cushion has a diameter of 30 mm. In some embodiments, the cushion has a diameter of 35 mm. In some embodiments, the cushion has a diameter of 38 mm.

Without intending to be limited to any particular theory, the skin-electrode impedance varies with the pressure at which the electrode contacts the skin of the pregnant human subject. In some embodiments, the skin-electrode impedance decreases as the pressure at which the electrode contacts the skin of the pregnant human subject increases. In some embodiments, the pressure is applied using a garment, such as a belt.

In some embodiments, the suitable skin-electrode impedance is between 100 and 650 kΩ. In some embodiments, the suitable skin-electrode impedance is 602 kΩ. In some embodiments, the suitable skin-electrode impedance is less than 150 kΩ. In some embodiments, the suitable skin-electrode impedance is 227 kΩ. In some embodiments, the suitable skin-electrode impedance is 135 kΩ.

In some embodiments, an exemplary electrode (e.g., any of the electrodes 100, 200, 400 described above) is configured to have skin-electrode impedance of less than 150 kΩ. In some embodiments, an exemplary electrode is configured to have skin-electrode impedance of between 5 to 150 kΩ. In some embodiments, an exemplary electrode is configured to have skin-electrode impedance of between 10 to 150 kΩ. In some embodiments, an exemplary electrode is configured to have skin-electrode impedance of between 20 to 150 kΩ. In some embodiments, an exemplary electrode is configured to have skin-electrode impedance of between 30 to 150 kΩ. In some embodiments, an exemplary electrode is configured to have skin-electrode impedance of between 40 to 150 kΩ. In some embodiments, an exemplary electrode is configured to have skin-electrode impedance of between 50 to 150 kΩ. In some embodiments, an exemplary electrode is configured to have skin-electrode impedance of between 60 to 150 kΩ. In some embodiments, an exemplary electrode is configured to have skin-electrode impedance of between 70 to 150 kΩ. In some embodiments, an exemplary electrode is configured to have skin-electrode impedance of between 80 to 150 kΩ. In some embodiments, an exemplary electrode is configured to have skin-electrode impedance of between 90 to 150 kΩ. In some embodiments, an exemplary electrode is configured to have skin-electrode impedance of between 100 to 150 kΩ. In some embodiments, an exemplary electrode is configured to have skin-electrode impedance of between 110 to 150 kΩ. In some embodiments, an exemplary electrode is configured to have skin-electrode impedance of between 120 to 150 kΩ. In some embodiments, an exemplary electrode is configured to have skin-electrode impedance of between 130 to 150 kΩ. In some embodiments, an exemplary electrode is configured to have skin-electrode impedance of between 140 to 150 kΩ. In some embodiments, after gel has been applied to an exemplary electrode, the skin-electrode impedance is between 5 and 150 kΩ.

In some embodiments, an exemplary electrode (e.g., any of the electrodes 100, 200, 400 described above) includes an elastomeric structure that is configured to deform when placed on the abdomen of the pregnant human subject. In some embodiments, the deformation of the elastomeric structure creates a frictional engagement between the elastomeric structure and the skin, thereby preventing displacement of the elastomeric structure (and, thereby, the electrode as a whole) due to events such as muscular activity, fetal kicks, etc. In some embodiments, by maintaining the electrode in a static position, a constant skin-electrode impedance can be maintained. Additionally, in some embodiments, the deformation of the elastomeric structure causes the elastomeric structure to press against the skin around the entire perimeter of the cavity, thereby preventing the gel from running out of the cavity and from drying.

In some embodiments, an exemplary electrode (e.g., any of the electrodes 100, 200, 400 described above) is configured to have a capacitance suitable for sensing fetal electrocardiogram signals from a pregnant human subject. In some embodiments, the capacitance is from 1 nF to 0.5 μF. In some embodiments, the capacitance is 5 nF. In some embodiments, the capacitance is 10 nF. In some embodiments, the capacitance is 15 nF. In some embodiments, the capacitance is 20 nF. In some embodiments, the capacitance is 25 nF. In some embodiments, the capacitance is 30 nF. In some embodiments, the capacitance is 35 nF. In some embodiments, the capacitance is 40 nF. In some embodiments, the capacitance is 45 nF. In some embodiments, the capacitance is 50 nF. In some embodiments, the capacitance is 60 nF. In some embodiments, the capacitance is 70 nF. In some embodiments, the capacitance is 80 nF. In some embodiments, the capacitance is 90 nF. In some embodiments, the capacitance is 95 nF. In some embodiments, the capacitance is 0.1 μF.

Without intending to be limited to any particular theory, the capacitance of the electrodes increases as the surface area of the cutaneous contact that contacts the skin of the pregnant human subject increases. Additionally, without intending to be limited to any particular theory, the capacitance of the electrodes decreases as the pressure applied to the cutaneous contact increases.

In some embodiments, the electrode is configured to detect a fetal electrocardiogram signal having a signal to noise ratio between −20 dB and 50 dB. In some embodiments, the electrode is configured to detect a fetal electrocardiogram signal having a signal to noise ratio between 0 dB and 50 dB. In some embodiments, the electrode is configured to detect a fetal electrocardiogram signal having a signal to noise ratio less than 50 dB.

### Systems for Sensing Fetal Cardiac Electrical Activity

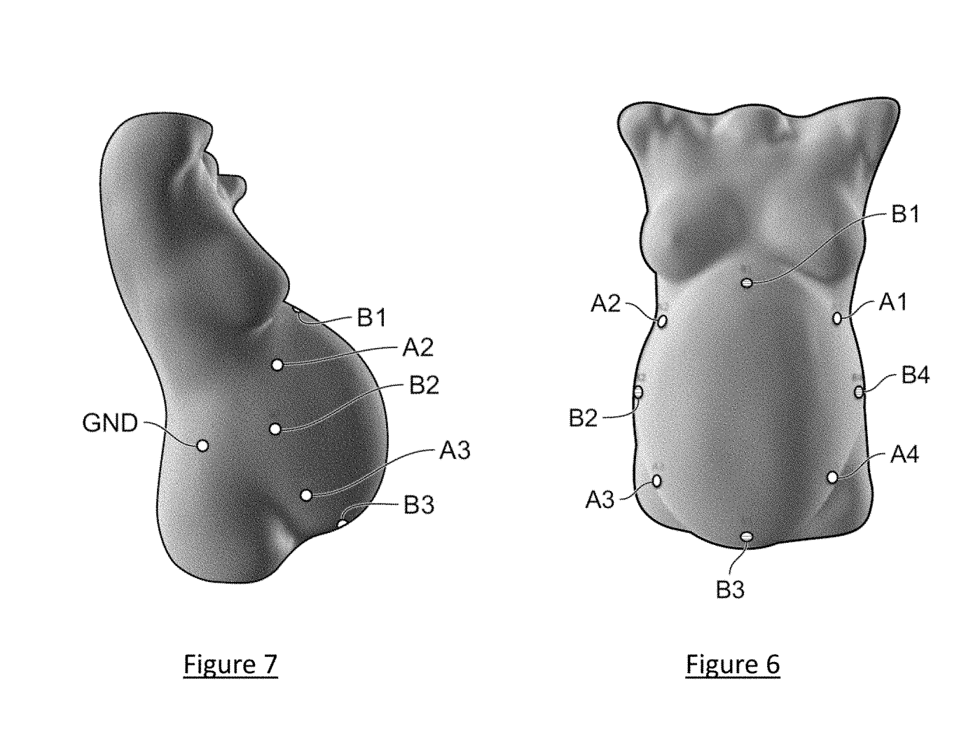

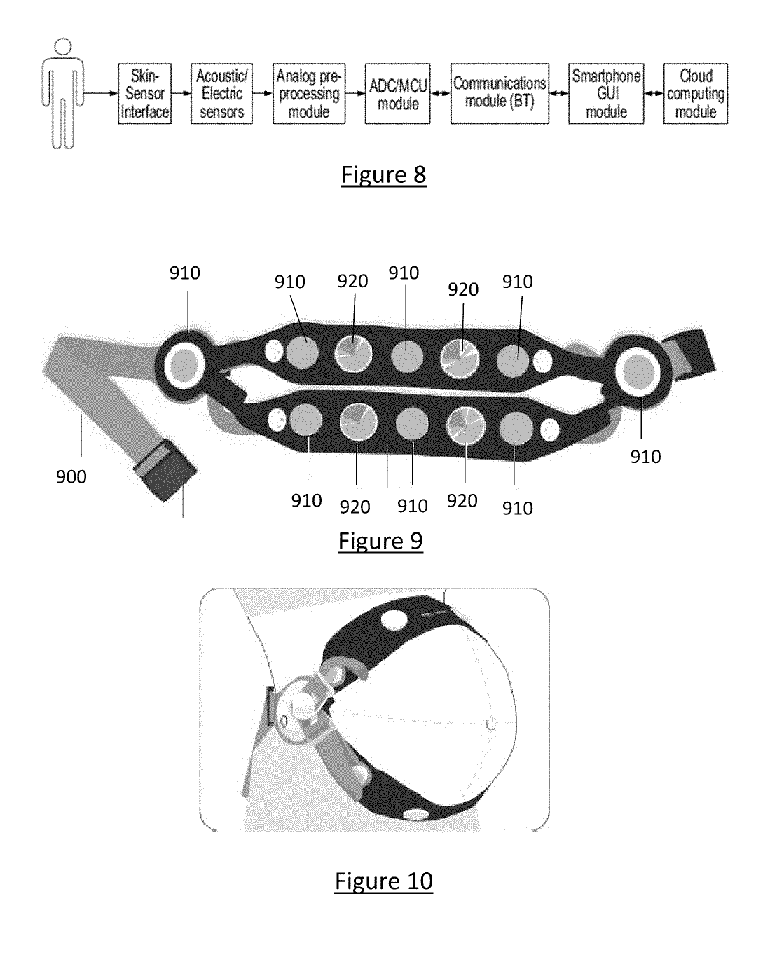

In some embodiments, the arrangement of the electrodes provides a system for recording, detecting and analyzing fetal cardiac electrical activity data regardless of sensor position, fetal orientation, fetal movement, or gestational age. In some embodiments, the electrodes are attached, or positioned, on the abdomen of the pregnant human subject in the configuration shown in FIGS. 6 and 7. In some embodiments, the electrodes are divided into channels comprising a pair of electrodes, and cardiac electrical activity data is recorded simultaneously from the channels. In some embodiments, the channels output the acquired signal data, corresponding to the recorded cardiac electrical activity data.

Referring to FIG. 8, in some embodiments, the system for recording, detecting and analyzing fetal cardiac electrical activity comprises a skin-electrode interface, at least one electrode, an analog pre-processing module, an analog to digital converter/microcontroller (ADC/MCU) module, a communications module, a smartphone module, and a cloud computing module.

In some embodiments, the analog pre-processing module performs at least one function selected from the group consisting of: amplification of the recorded signals, and filtering the recorded signals.

In some embodiments, the ADC/MCU module performs at least one task selected from the group consisting of: converting analog signals to digital signals, converting the recorded signals to digital signals, compressing the data, digital filtering, and transferring the recorded electrocardiogram signals data to the transmitter.

In some embodiments, the communications module transmits the recorded signals to a wireless receiver.

In some embodiments, recording, detecting and analyzing fetal cardiac electrical activity data regardless of sensor position, fetal orientation, fetal movement, or gestational age may be performed in accordance with one or more techniques disclosed in U.S. Pat. No. 9,572,504.

In some embodiments, at least one electrode pair is used to obtain the acquired signal data. In some embodiments, For example, by way of a non-limiting illustration, in some embodiments, the channels are specified as follows:

1. A1-A4

2. A2-A3

3. A2-A4

4. A4-A3

5. B1-B3

6. B1-B2

7. B3-B2

8. A1-A3

In some embodiments, the signal data corresponding to fetal cardiac electrical activity data are extracted from the acquired signal data.

In some embodiments, signal data corresponding to fetal cardiac electrical activity data may be extracted from the acquired signal data in accordance with one or more techniques disclosed in U.S. Pat. No. 9,392,952.

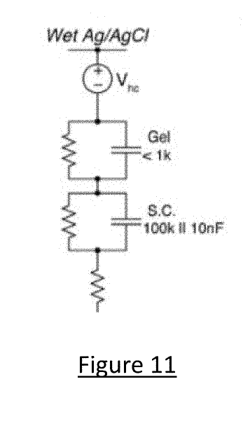

Referring to FIG. 9, an example of a garment 900 according to some embodiments of the present invention is shown. In some embodiments, the garment 900 is a belt. In the embodiment shown, eight (8) electrodes 910 (e.g., any of the exemplary electrodes described above) are incorporated into a belt, wherein the belt, when worn, positions the electrodes 910 on the abdomen of the pregnant mother, such that the electrodes 910 contact the skin of the abdomen of the pregnant mother, and the electrodes 910 are positioned in a circumferential arrangement around the uterus. In some embodiments, the belt also contains additional sensors 920 and a transmitter. In some embodiments, the additional sensors 920 are acoustic sensors. Referring to FIG. 10, the garment 900 is shown as worn by a pregnant mother.

Reference is now made to the following examples, which together with the above descriptions illustrate some embodiments of the invention in a non-limiting fashion.

### EXAMPLES

### Example 1: Electrophysiological Performance Measurements

The source of the physiological signals detected using the electrodes according to some embodiments of the present invention are located within the body of the pregnant human subject and have extremely low amplitude and low frequency. Without intending to be limited by any particular theory, the physiological signals flow within the body of the pregnant human subject by the movement of ions. The electrodes according to some embodiments of the present invention act as signal transducers, and transduce the movement of ions to the movements of electrons. The skin-electrode interface is one determining factor of the electrode's ability to transduce the physiological signals.

The skin-electrode interface for the electrodes according to some embodiments of the present invention may be modeled by a parallel circuit of an ohmic and capacitive impedance with an additional Warburg resistance (see FIG. 11). Without intending to be limited to any particular theory, both the conductive and the capacitive portions of the equivalent circuit affect the performance of an electrode according to some embodiments of the present invention. The skin-electrode impedance is equivalent to the impedance of the circuit shown in FIG. 11, and, in various situations, may range from 10 kΩ to 100 MΩ. Without intending to be limited to any particular theory, decreasing the impedance improves the performance of an electrode according to some embodiments of the present invention. In some embodiments, decreasing impedance may be achieved by increasing the size of the contact area, or by reducing the resistivity of the cutaneous contact. An increase in input impedance and a decrease in input capacitance of the amplifier may also improve the performance of an electrode according to some embodiments of the present invention.

### Example 2: Electrodes According to Some Embodiments of the Present Invention

Various electrodes were manufactured according to the embodiment shown in FIG. 1 and evaluated. Two such electrodes were incorporated into a garment as described above with reference to FIG. 9, and two reference electrodes were provided. The reference electrodes were the electrodes commercialized under the trade name AMBU WHITESENSOR 4500M-H by Ambu A/S of Ballerup, Denmark. An ECG gel was applied to the experimental electrodes, and the garment was donned by a pregnant human subject. The reference electrodes were affixed to the subject's body adjacent to the experimental electrodes. Signal voltage amplitude was recorded for both the experimental electrodes and the reference electrodes over a period of 1.5 minutes, and the experimental electrodes were deemed to be passing if the median recorded signal voltage was greater than or equal to 85% of the median recorded signal voltage.

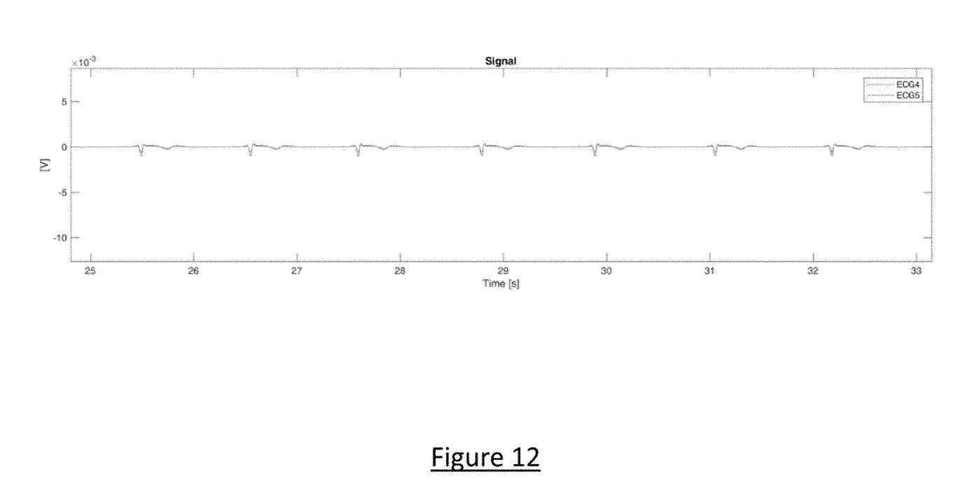

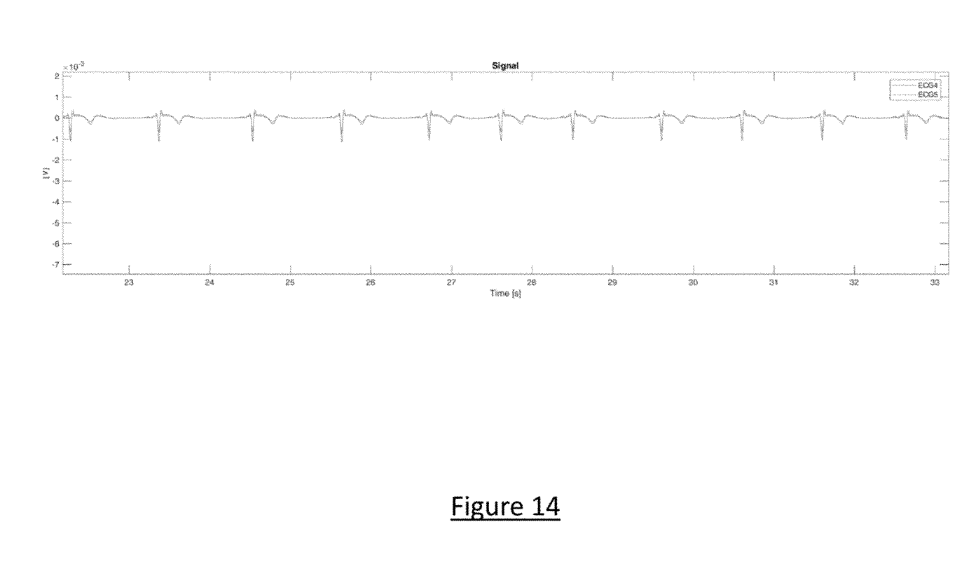

FIGS. 12-14 show plots comparing data recorded by the experimental electrodes to data recorded by the reference electrodes, as noted above. In each of FIGS. 12-14, the legend “ECG4” denotes the plot showing data recorded by the experimental electrodes, while the legend “ECG5” denotes the plot showing data recorded by the reference electrodes. The relative performance of the experimental electrodes and the reference electrodes is summarized in Table 1 below.

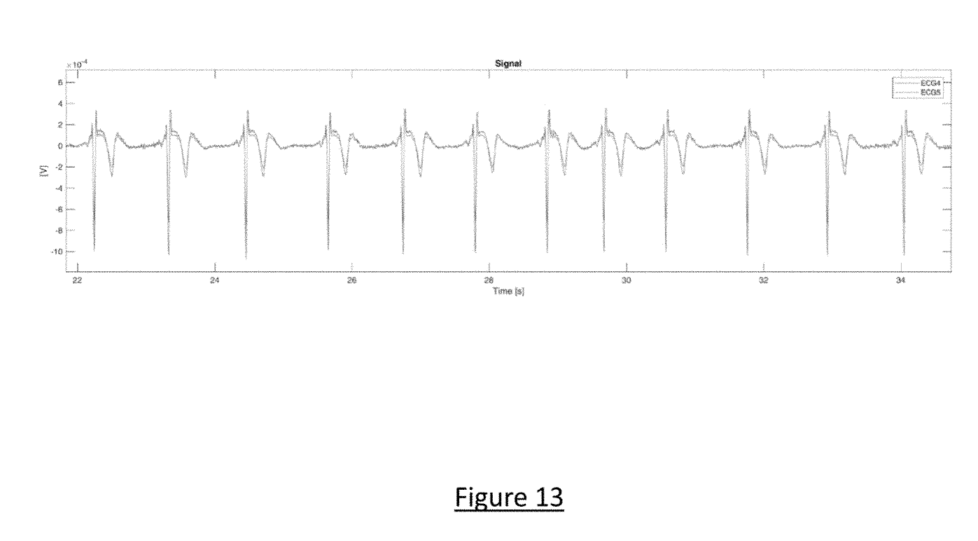

TABLE 1

| | | Reference | Experimental | Ratio of | Experimental |
|---|---|---|---|---|---|
| Measurement | | Sensor | Sensor | Experimental | >85% of |
| Number | FIG. | Median (mV) | Median (mV) | to Reference | Reference? |
| 1 | FIG. 11 | 0.58 | 0.97 | 168.2281 | Pass |
| 2 | FIG. 12 | 0.69 | 0.98 | 142.6087 | Pass |
| 3 | FIG. 13 | 0.64 | 1.04 | 163.0469 | Pass |

As may be seen, the experimental sensors met the criteria for passing tests, and, further, compared favorably to the reference sensors.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

What is claimed is:

1. An electrode, comprising:
(a) a cutaneous contact for sensing electrocardiogram signals from a pregnant human subject, the electrocardiogram signals containing fetal electrocardiogram signals;
(b) a connector in electrical contact with the cutaneous contact for transmission of the electrocardiogram signals from the cutaneous contact to a destination; and
(c) a cushion including a cavity, the cushion being configured such that the cavity faces the pregnant human subject when the electrode is in use,
wherein the cutaneous contact is coupled to the cushion such that the cutaneous contact is positioned within the cavity, the cushion and the cutaneous contact configured to allow a surface of the cutaneous contact to be an electrical interface with a skin of the pregnant human subject when the electrode is in use, and
wherein the cavity is configured so as to receive and retain therein an amount of a conductive wetting substance that is sufficient to provide a skin-electrode impedance of less than 150 kΩ when the electrocardiogram signals are at frequencies of 10 Hz or less.

2. The electrode of claim 1, wherein the cutaneous contact comprises silver.

3. The electrode of claim 1, wherein the skin-electrode impedance is between 5 kΩ and 150 kΩ at frequencies of 10 Hz and below.

4. The electrode of claim 1, wherein the electrode provides a signal-to-noise ratio of fetal electrocardiogram signals that is less than 50 dB.

5. The electrode of claim 1, further comprising:
a button snap stud having a flange portion; and
a button snap post having a flange portion, the button snap post being configured to snap into the button snap stud,
wherein the cushion includes a lip surrounding a central bore, the central bore being sized such that the flange portion of the button snap stud and the flange portion of the button snap post cannot pass through the central bore,
wherein the button snap post is snapped into the button snap stud in respective positions such that flange portion of the button snap post and the flange portion of the button snap stud are positioned on opposite sides of the lip of the cushion so as to fix the button snap post and the button snap stud to the cushion, and
wherein the cutaneous contact is retained by the button snap post.

6. The electrode of claim 5, further comprising:
a frame comprising a plastic material, wherein the cushion encompasses the frame.

7. The electrode of claim 1, wherein the cavity is circular and has an opening diameter at an opening where the cushion contacts the skin of the pregnant human subject, and wherein the opening diameter is between 12 mm and 17 mm.

8. The electrode of claim 1, wherein the cavity has a base diameter at a base opposite the opening, and wherein the base diameter is between 9 mm and 11 mm.

9. The electrode of claim 1, wherein the cavity has a depth of between 0.2 mm and 4.5 mm.

10. The electrode of claim 1, wherein the electrode meets at least one criterion of an ANSI/AAMI EC12:2000/®2015 standard for Disposable ECG Electrodes.

11. A kit, comprising:
a conductive wetting substance; and
at least one electrode configured to detect fetal electrocardiogram signals through the use of the wetting substance, the electrode comprising:
(a) a cutaneous contact for sensing electrocardiogram signals from a pregnant human subject, the electrocardiogram signals containing fetal electrocardiogram signals;
(b) a connector in electrical contact with the cutaneous contact for transmission of the electrocardiogram signals from the cutaneous contact to a destination; and
(c) a cushion including a cavity, the cushion being configured such that the cavity faces the pregnant human subject when the electrode is in use,
wherein the cutaneous contact is coupled to the cushion such that the cutaneous contact is positioned within the cavity, the cushion and the cutaneous contact configured to allow a surface of the cutaneous contact to be an electrical interface with a skin of the pregnant human subject when the electrode is in use, and
wherein the cavity is configured so as to receive and retain therein an amount of a conductive wetting substance that is sufficient to provide a skin-electrode impedance of less than 150 kΩ when the electrocardiogram signals are at frequencies of 10 Hz or less.

12. The kit of claim 11, wherein the wetting substance is a conductive gel.

13. The kit of claim 12, wherein the conductive gel has an electrical length admittance of between 3.6 and 313 μS/cm at a frequency of 1 Hz

14. The kit of claim 11, wherein the wetting substance is a conductive paste.

15. The kit of claim 14, wherein the conductive paste has an electrical length admittance of between 250 and 300 μS/cm at a frequency of 1 Hz

16. A garment, comprising:
at least one pair of electrodes, each of the electrodes being configured to detect fetal electrocardiogram signals through the use of a wetting substance, each of the electrodes comprising:
(a) a cutaneous contact for sensing electrocardiogram signals from a pregnant human subject, the electrocardiogram signals containing fetal electrocardiogram signals;
(b) a connector in electrical contact with the cutaneous contact for transmission of the electrocardiogram signals from the cutaneous contact to a destination; and
(c) a cushion including a cavity, the cushion being configured such that the cavity faces the pregnant human subject when the electrode is in use,
wherein the cutaneous contact is coupled to the cushion such that the cutaneous contact is positioned within the cavity, the cushion and the cutaneous contact configured to allow a surface of the cutaneous contact to be an electrical interface with a skin of the pregnant human subject when the electrode is in use, and
wherein the cavity is configured so as to receive and retain therein an amount of a conductive wetting substance that is sufficient to provide a skin-electrode impedance of less than 150 kΩ when the electrocardiogram signals are at frequencies of 10 Hz or less.

17. The garment of claim 16, wherein the garment includes a belt.

18. The garment of claim 16, further comprising at least one acoustic sensor.

19. The garment of claim 16, wherein the at least one pair of electrodes comprises eight electrodes.

20. The garment of claim 19, wherein the electrodes are positioned within the garment such that, when the garment is worn by the pregnant human subject, the electrodes are positioned in a circumferential arrangement around a uterus of the pregnant human subject.
